SEQUENCE LISTING (46 sequences)

SEQ ID NO: 1 - DNA, 26 bp, Artificial Sequence
  Primer
taataatttatgtataaaattaaggg

SEQ ID NO: 2 - DNA, 23 bp, Artificial Sequence
  Primer
atccaaatatctttacttttctt

SEQ ID NO: 3 - DNA, 25 bp, Artificial Sequence
  Primer
tgttaaaagttattgtgttttgaag

SEQ ID NO: 4 - DNA, 24 bp, Artificial Sequence
  Primer
atccaactaaaccatctatttcat

SEQ ID NO: 5 - DNA, 22 bp, Artificial Sequence
  Primer
atgaaatagatggtttagttgg

SEQ ID NO: 6 - DNA, 24 bp, Artificial Sequence
  Primer
tcatctaatataacatcccctatt

SEQ ID NO: 7 - DNA, 22 bp, Artificial Sequence
  Primer
ataggggatgttatattagatg

SEQ ID NO: 8 - DNA, 22 bp, Artificial Sequence
  Primer
aatatatctttcactaacaccc

SEQ ID NO: 9 - DNA, 27 bp, Artificial Sequence
  Primer
ttagtaatgtaaaggtagtaatgttag

SEQ ID NO: 10 - DNA, 21 bp, Artificial Sequence
  Primer
ttccccataaacatactaaac

SEQ ID NO: 11 - DNA, 21 bp, Artificial Sequence
  Primer
gtgtttttaatgtgtatgatg

SEQ ID NO: 12 - DNA, 26 bp, Artificial Sequence
  Primer
tacctatattaactacaccatataat

SEQ ID NO: 13 - DNA, 22 bp, Artificial Sequence
  Primer
tgttggtatagattttaggtgg

SEQ ID NO: 14 - DNA, 23 bp, Artificial Sequence
  Primer
caaccaatattaacaccacttaa

SEQ ID NO: 15 - DNA, 22 bp, Artificial Sequence
  Primer
ataaggttagagaaatgggatt

SEQ ID NO: 16 - DNA, 23 bp, Artificial Sequence
  Primer
aaccctctaccacaattactaat

SEQ ID NO: 17 - DNA, 22 bp, Artificial Sequence
  Primer
tagtggaagtgtagtttgatgg

SEQ ID NO: 18 - DNA, 27 bp, Artificial Sequence
  Primer
cactatccactaaatctctatacaaca

SEQ ID NO: 19 - DNA, 26 bp, Artificial Sequence
  Primer
aagttgttgtatagagatttagtgga

SEQ ID NO: 20 - DNA, 22 bp, Artificial Sequence
  Primer
taaacaaacacacaaaaacaca

SEQ ID NO: 21 - DNA, 22 bp, Artificial Sequence
  Primer
ttttgtgtgtttttgtgtgttt

SEQ ID NO: 22 - DNA, 22 bp, Artificial Sequence
  Primer
aaacacctaaacrcaaaaacta

SEQ ID NO: 23 - DNA, 22 bp, Artificial Sequence
  Primer
tagtagtttttgygtttaggtg

SEQ ID NO: 24 - DNA, 24 bp, Artificial Sequence
  Primer
ttcaacaataattttaccttcaac

SEQ ID NO: 25 - DNA, 23 bp, Artificial Sequence
  Primer
ggttgaaggtaaaattattgttg

SEQ ID NO: 26 - DNA, 25 bp, Artificial Sequence
  Primer
acaccaacatcaataaaactaattt

SEQ ID NO: 27 - DNA, 28 bp, Artificial Sequence
  Primer
agtaattagtagtatatttatattaggg

SEQ ID NO: 28 - DNA, 27 bp, Artificial Sequence
  Primer
atcataataataatataccttaacacc

SEQ ID NO: 29 - DNA, 27 bp, Artificial Sequence
  Primer
ggattatatgatatttatgtagatgat

SEQ ID NO: 30 - DNA, 27 bp, Artificial Sequence
  Primer
atccaactacaaataatctaaatattc

SEQ ID NO: 31 - DNA, 26 bp, Artificial Sequence
  Primer
taaagtattaggattataatataggg

SEQ ID NO: 32 - DNA, 23 bp, Artificial Sequence
  Primer
tctattatccacacctacattta

SEQ ID NO: 33 - DNA, 22 bp, Artificial Sequence
  Primer
ggttttggtgttatggatttta

SEQ ID NO: 34 - DNA, 22 bp, Artificial Sequence
  Primer
ttaattaccccaacaaatacca

SEQ ID NO: 35 - DNA, 22 bp, Artificial Sequence
  Primer
aaaggttttgggtttattgtaa

SEQ ID NO: 36 - DNA, 22 bp, Artificial Sequence
  Primer
attccaatcctccaaaataata

SEQ ID NO: 37 - DNA, 24 bp, Artificial Sequence
  Primer
aaaggaaaagttttttgtagattt

SEQ ID NO: 38 - DNA, 26 bp, Artificial Sequence
  Primer
taaataaccacaacacaattaataaa

SEQ ID NO: 39 - DNA, 24 bp, Artificial Sequence
  Primer
aggattgaaggttaaattaaaatt

SEQ ID NO: 40 - DNA, 23 bp, Artificial Sequence
  Primer
aacacattttataccaaaaaaca

SEQ ID NO: 41 - DNA, 23 bp, Artificial Sequence
  Primer
tgttttttggtataaaatgtgtt

SEQ ID NO: 42 - DNA, 22 bp, Artificial Sequence
  Primer
cacacacccatatacaatttta

SEQ ID NO: 43 - DNA, 24 bp, Artificial Sequence
  Primer
tgcgatataaacgttttgtaaaac

SEQ ID NO: 44 - DNA, 20 bp, Artificial Sequence
  Primer
aatatacccaatacgtccgc

SEQ ID NO: 45 - DNA, 27 bp, Artificial Sequence
  Primer
taatgtgatataaatgttttgtaaaat

SEQ ID NO: 46 - DNA, 23 bp, Artificial Sequence
  Primer
aaatatacccaatacatccacct

(19) United States
(12) Patent Application Publication
(10) Pub. No.: US 2010/0285443 A1
(43) Pub. Date: Nov. 11, 2010

(54) Diagnostic Methods for Diseases Caused by a HPV Infection Comprising Determining the Methylation Status of the HPV Genome

(75) Inventor: Manel Esteller, Madrid (ES)

Correspondence Address: ANDRUS, SCEALES, STARKE & SAWALL, LLP, 100 EAST WISCONSIN AVENUE, SUITE 1100, MILWAUKEE, WI, 53202, US

(73) Assignee: OncoMethylome Sciences SA, Sart-Tilman (BE)

(21) Appl. No.: 12/519,268

(22) PCT Filed: Dec. 17, 2007
(86) PCT No.: PCT/GB2007/004845

§ 371 Date: Mar. 31, 2010

Related U.S. Application Data
(60) Provisional application No. 60/874,958, filed on Dec. 15, 2006.

(51) Int. Cl. C12Q 1/70; C12Q 1/68; C07H 21/04

(52) U.S. Cl. 435/5; 435/6; 536/24.33

(57) ABSTRACT

Methods and kits for diagnosing and/or monitoring the progression of or otherwise staging a disease caused by a human papillomavirus (HPV) infection in a test sample obtained from a subject comprise determining the methylation status of a HPV genome. The presence of hypermethylation of the HPV genome indicates a positive diagnosis of the disease and/or an increased level of methylation of the HPV genome indicates the progression of the disease to a more advanced form. Suitable diseases linked to HPV infection include cancers such as cervical cancer. High risk HPV types such as HPV16 are generally assessed in the methods and using the kits of the invention. The HPV16 methylome is provided.

### FIELD OF THE INVENTION

The invention relates to diseases linked to viral infections and in particular to diseases associated with human papillomavirus (HPV) infections. Methods and kits are provided for diagnosing, staging or otherwise monitoring the progress of such diseases. These methods and kits generally involve determining the methylation status of at least a portion of a HPV genome.

### BACKGROUND TO THE INVENTION

It has been estimated by the World Health Organization that 15% of cancer cases are etiologically linked to viral infection; accounting for nearly 1.5 million new cases and 900,000 deaths annually worldwide. Some viruses have been linked to specific forms of cancer, and even proteins from non-tumorigenic viruses in humans can cause immortalization of cells in culture and tumours in transgenic mice. Virus infection often triggers the host defense mechanism to silence the foreign genome, especially in DNA viruses which can insert themselves into the host genome. (Butel, 2000; Gatza, 2005; Li, 2005).

Human Papilloma viruses (HPV) are small, non-enveloped viruses that infect the cutaneous and mucosal epithelium and are widespread within humans. They are responsible for a number of benign diseases such as warts and verrucas, but they are also associated with more serious conditions as cervical, skin and oral cancer. It is in cervical cancer where these viruses have been more extensively studied. Cancer of the cervix is the second most frequent gynecological malignancy in women worldwide. Particularly in Europe each year 44,000 women develop cervical cancer and 23,000 women die from this disease. Infection with certain types of the human papilloma virus family is the primary risk factor for the development of this type of neoplasm and its precursor lesions. The high-risk HPV-16 and 18 can be identified in 50-60% and 10-20% of cervical tumour samples respectively (Bosch, 2001; Davies, 2006; Gillison, 2004; Hebner, 2006, Pfister, 2003).

HPV 16 from the genus alpha-papillomavirus, has circular doubled-stranded DNA with size close to 8 kb. In the course of cancer development, the viral molecule frequently becomes integrated into host-cell DNA and some parts of the viral genome are frequently deleted. Some HPV genes possess proliferation-stimulating properties as is the case of E5, E6 and E7; but only E6 and E7 seem to have a significant role for HPV related malignant transformation. Several studies have shown that the expression of these oncoproteins is required as well for maintaining the malignant growth of cervical cancer cells, particularly activating cyclins E and A, and inhibiting the p53 and RB tumour suppressors. Other viral genes modulate transcription and replication (in the case of E1 and E2) and finally two structural proteins compose the viral capsids (L1 and L2) (De Villiers, 2004; Zur Hausen, 2002).

Related to cervical carcinoma, the infection of HPV, commonly transmitted by sexual contact, is closely linked to epithelial differentiation (FIG. 1A). It requires the availability of epidermal or mucosal epithelial cells still able to proliferate, which are located in the basal layer. In this subpopulation of cells some early genes are expressed (such as E6 and E7) and the viral production continues while the cells progress up to the upper layers of the epidermis or mucosa, where complete viral particles are assembled and released. This infection results initially in lesions that are cleared 6-12 months after appearance, probably by immunological mechanisms. However, sometimes these lesions progress to a carcinoma in situ and, if not surgically intervened, to squamous-cell carcinoma or adenocarcinoma of the cervix (FIG. 1B) (Lowy, 2006; Zur Hausen, 2002).

The factors that determine the progression of subclinical lesion to invasive carcinoma are poorly understood. In the development of cancer, tumour suppressor genes are inactivated by a number of processes including promoter hypermethylation. This epigenetic process consists in the addition of a methyl group in the cytosine residue of the gene promoter CpG island, with a concomitant recruitment of specific proteins, and compaction of chromatin, thus consequently gene transcription is inhibited or silenced (FIG. 2). In the case of cervical carcinogenesis, aberrant methylation of CpG islands within the promoter regions of tumour suppressor genes starts early in the carcinogenesis process and increases with the pathology of the lesions. It has been reported that methylation of RARβ and GSTP1 are early events, p16 and MGMT are intermediate events, and FHIT methylation is a late event which is associated with cervical carcinomas (Virmani, 2001; Dong, 2001).

The cellular CpG methylation machinery also efficiently targets the viral genome infecting the cell. This host's genome defense hypothesis is based on the frequent methylation of chromosomally integrated exogenous genes (such as transgenes and transfected adenoviral genomes). Surprisingly, viruses can find ways to counteract this cell-based modification and even take advantages of it, as it would allow them to establish a persistent infection and help them to evade the host immune system.

In the case of papilloma viruses, it has been reported that one gene in HPV-18 (the L1 gene), using bisulfite sequencing in a small number of clinical samples, was commonly methylated in all cases, and it could be a biomarker of neoplastic progression as it was discovered hypomethylated in asymptomatic patients and precancerous lesions; and hypermethylated in carcinomas. Related to HPV-16, it has been shown (looking to only a small portion of the genome) to be heavily methylated in cancers and low methylated in CIN lesions (Turan, 2006; Badal, 2004; Kalantari, 2004).

### DESCRIPTION OF THE INVENTION

In a first aspect, the invention provides a method of diagnosing and/or monitoring the progression of or otherwise staging a disease caused by a human papillomavirus (HPV) infection in a test sample obtained from a subject comprising determining the methylation status of a HPV genome wherein the presence of hypermethylation of the HPV genome indicates a positive diagnosis of the disease and/or an increased level of methylation of the HPV genome indicates the progression of the disease to a more advanced form.

The methods of the invention are most preferably ex vivo or in vitro methods carried out on a test sample. In one embodiment the method may also include the step of obtaining the sample. The methods can be used to diagnose any disease caused by, or resulting from, infection by a human papillomavirus (HPV). HPV infections contribute to a number of conditions, ranging from warts and verrucas to more serious diseases such as cancer. The methods of the invention may, therefore, be used in a range of applications. In one embodiment, the disease which is diagnosed, staged or otherwise monitored comprises, consists essentially of or consists of cancer. HPV infections may contribute to the incidence of a range of cancers. Thus, in specific embodiments, the cancer is selected from anogenital cancers or skin cancers. The cancer may be cervical cancer, skin cancer or head and neck cancer for example.

In a specific embodiment, the methods of the invention are utilised in order to stage the disease into one of several categories of varying severity. Thus the progression of the disease may be monitored having regard to the (relative) methylation status of the HPV genome in the sample. The categories may be, for example HPV carrier, pre-malignancy or primary tumour. “HPV carrier” indicates an asymptomatic subject infected with the virus. By “asymptomatic” is meant that clinical symptoms are not displayed by the subject. “Pre-malignancy” indicates subjects who are suffering from pre-tumourigenic lesions, or neoplasias. “Primary tumour” indicates the presence of a tumourigenic lesion. These categories are linked to the methylation status of the HPV genome. Generally, hypomethylation indicates a HPV carrier and hypermethylation indicates a primary tumour. An intermediate methylation status between hypomethylation and hypermethylation may indicate pre-malignancy. Hypomethylation and Hypermethylation are well known terms in the art to describe (relatively) under and over methylated regions of the genome respectively.

In a specific embodiment, where the cancer is cervical cancer, the pre-malignancy may comprise, consist essentially of or consist of cervical intraepithelial carcinoma. Also where the cancer is cervical cancer, the primary tumour may comprise, consist essentially of or consist of squamous cell carcinoma. The presence of hypermethylation of the HPV genome may be used as an indication of a positive diagnosis of squamous cell carcinoma. In a related embodiment, the presence of hypermethylation of the HPV genome indicates a positive diagnosis of squamous cell carcinoma and the presence of less methylation/relative hypomethylation indicates the presence of cervical intraepithelial neoplasia. Thus, in one aspect the invention provides a method of diagnosing squamous cell carcinoma in a test sample obtained from a subject comprising determining the methylation status of a HPV-16 genome wherein the presence of hypermethylation of the HPV-16 genome indicates a positive diagnosis of squamous cell carcinoma. The invention also provides a method of distinguishing squamous cell carcinoma and cervical intraepithelial neoplasia in a test sample obtained from a subject comprising determining the methylation status of a HPV-16 genome wherein the presence of hypermethylation of the HPV-16 genome indicates the presence of squamous cell carcinoma, whereas hypomethylation of the HPV-16 genome indicates the present of cervical intraepithelial neoplasia.

The “test sample” may comprise, consist essentially of or consist of any suitable tissue sample or body fluid. A suitable test sample is thus one representative of the presence of the HPV genome and in which the methylation status may be determined. Preferably, the test sample is obtained from a human subject. The type of sample which is appropriate can be readily determined by the nature of the disease to be diagnosed, staged or otherwise monitored according to the methods of the invention. Thus, for example, if the disease to be investigated is cervical cancer, the sample may be a suitable cervical sample. Suitable samples may be obtained by any method. For example, a suitable cervical sample may be obtained by a cervical scraping.

Other DNA-containing samples which may be used in the methods of the invention include samples for diagnostic, prognostic, or personalised medicinal uses provided that they contain the HPV genome (or at least a suitable portion thereof) for investigation of its methylation status as an indication of the disease. These samples may be obtained from surgical samples, such as biopsies or fine needle aspirates, from paraffin embedded tissues, from frozen tumor tissue samples, from fresh tumor tissue samples or from a fresh or frozen body fluid, for example. Non-limiting examples include whole blood or parts/fractions thereof, bone marrow, cerebrospinal fluid, peritoneal fluid, pleural fluid, lymph fluid, serum, plasma, urine, chyle, ejaculate, sputum, nipple aspirate, saliva, swabs specimens, colon wash specimens and brush specimens. The tissues and body fluids can be collected using any suitable method. Many such methods are well known in the art. Assessment of a paraffin-embedded specimen can be performed directly or on a tissue section.

“Diagnosis” is defined herein to include screening for a disease or pre-indication of a disease, identifying a disease or pre-indication of a disease, monitoring the staging and the state and progression of the disease, checking for recurrence of disease following treatment and monitoring the success of a particular treatment. The methods of the invention may also have prognostic value, and this is included within the definition of the term “diagnosis”. The prognostic value of the methods of the invention may be used as a marker of potential susceptibility to the disease associated with a HPV infection or as a marker for progression of the disease, for example from pre-malignancy to primary tumour, or from carrier to pre-malignancy for example. Thus patients at risk may be identified before the disease has a chance to manifest itself in terms of symptoms identifiable in the patient.

The methods of the invention may be carried out on purified or unpurified DNA-containing samples. However, in a preferred embodiment, DNA is isolated/extracted/purified from the sample. Any suitable DNA isolation technique may be utilised. Examples of purification techniques may be found in standard texts such as Molecular Cloning—A Laboratory Manual (Third Edition), Sambrook and Russell (see in particular Appendix 8 and Chapter 5 therein). In one embodiment, purification involves alcohol precipitation of DNA. Suitable alcohols include ethanol and isopropanol. Suitable purification techniques also include salt-based precipitation methods. Thus, in one specific embodiment the DNA purification technique comprises use of a high concentration of salt to precipitate contaminants. The salt may comprise, consist essentially of or consist of potassium acetate and/or ammonium acetate for example. The method may further include steps of removal of contaminants which have been precipitated, followed by recovery of DNA through alcohol precipitation.

In an alternative embodiment, the DNA purification technique is based upon use of organic solvents to extract contaminants from cell lysates. Thus, in one embodiment, the method comprises use of phenol, chloroform and isoamyl alcohol to extract the DNA. Suitable conditions are employed to ensure that the contaminants are separated into the organic phase and that DNA remains in the aqueous phase.

In specific embodiments of these purification techniques, extracted DNA is recovered through alcohol precipitation, such as ethanol or isopropanol precipitation.

Amplification of DNA (using PCR) from natural sources is often inhibited by co-purified contaminants and various methods adopted for DNA extraction from environmental samples are available and provide an alternative for isolating DNA from test samples. Appropriate commercially available kits for isolating DNA from a test sample may thus be employed in the methods of the invention.

The methods of the invention may also, as appropriate, incorporate quantification of isolated/extracted/purified DNA in the sample. Quantification of the DNA in the sample may be achieved using any suitable means. Quantitation of nucleic acids may, for example, be based upon use of a spectrophotometer, a fluorometer or a UV transilluminator. Examples of suitable techniques are described in standard texts such as Molecular Cloning—A Laboratory Manual (Third Edition), Sambrook and Russell (see in particular Appendix 8 therein). In one embodiment, kits such as the Picogreen® dsDNA quantitation kit available from Molecular Probes, Invitrogen may be employed to quantify the DNA.

Determining the methylation status of a HPV genome may be achieved by any suitable means. As discussed herein, the present invention provides the HPV-16 methylome for the first time. Thus, in one embodiment, the methylation status of the entire HPV genome is determined. By this is meant that a global assessment of HPV genome methylation levels is carried out. Every CpG found in the known HPV genome can thus be investigated. Equally, every CpG island in the HPV genome, as can be identified using known software for example, may be investigated (see for example The CpGPLOT software program (http://bioinfo.hku.hk/cgi-bin) and the CpG Island Searcher software (see http://cpgislands.usc.edu/ and Takai D, Jones P A. The CpG island searcher: a new WWW resource. In Silico Biol. 3, 235-240 (2003)). This may be the HPV-16 methylome in one embodiment. In a specific embodiment, the methylation status of all 110 CpG residues in the 7904 nucleotide HPV16 genome is determined. The primers of table 1 and SEQ ID NOs 1 to 42 define the regions of interest to be investigated within the HPV16 genome. Thus, the methods of the invention may comprise, consist essentially of or consist of determination of the methylation status of a plurality, up to all (110 CpG residues for the HPV16 genome) in individual increments, CpG residues contained within the nucleotide sequences which can be sequenced using the primers of table 1 and SEQ ID NOs 1 to 42, as defined herein. Reference can also be made to FIG. 3. Thus, in one embodiment the methylation patterns presented in FIG. 3 are utilised as a reference in order to facilitate the diagnosis. Other suitable controls are described herein. In alternative embodiments, the methylation status of at least 5, 10, 15, 20, 25, 30, 35 etc up to all (in individual increments) of the methylation sites may be investigated. In further embodiments, the methods of the invention comprise, consist essentially of or consist of determining the methylation status of the L2 gene of the HPV genome and/or determining the methylation status of the E2 binding sites in the upstream regulatory region. Again, this may be carried out in respect of the HPV-16 genome in a particular embodiment. Suitable primers defining the regions of the L2 gene which may be investigated are shown in table 2 and in SEQ ID NOs 43 to 46. Other regions of the genome may be investigated as required. Overall investigation of the entire genome may provide the most informative results.

Various methylation assay procedures are known in the art, and can be used in conjunction with the present invention. In specific embodiments, the methylation status is determined using a technique selected from bisulphite sequencing, methylation specific PCR (MSP), microarray-based techniques, real-time amplification techniques and COBRA, either alone or in combination. Certain techniques for analysis of DNA methylation rely on the inability of methylation-sensitive enzymes to cleave methylated cytosine residues. Others rely upon treatment of DNA samples with a reagent such as sodium bisulphite which converts unmethylated cytosine to a changed nucleotide which displays different base pairing properties to cytosine, such as uracil, while methylated cytosines are maintained (Furuichi et al., 1970). This conversion results in a change in the sequence of the original DNA which can then be detected through one or more of a plurality of techniques. Any suitable technique may be employed in the methods of the invention.

Specific examples of techniques useful in the methods of the invention comprise, consist essentially of or consist of: sequencing, methylation-specific PCR (MSP), melting curve methylation-specific PCR (McMS-PCR), MLPA with or without bisulfite treatment, QAMA (Zeschnigk et al, 2004), MSRE-PCR (Melnikov et al, 2005), MethyLight (Eads et al., 2000), ConLight-MSP (Rand et al., 2002), bisulfite conversion-specific methylation-specific PCR (BS-MSP) (Sasaki et al., 2003), COBRA (which relies upon use of restriction enzymes to reveal methylation dependent sequence differences in PCR products of sodium bisulfite-treated DNA), methylation-sensitive single-nucleotide primer extension conformation (MS-SNuPE), methylation-sensitive single-strand conformation analysis (MS-SSCA), Melting curve combined bisulfite restriction analysis (McCOBRA) (Akey et al., 2002), PyroMethA, HeavyMethyl (Cottrell et al. 2004), MALDI-TOF, MassARRAY, Quantitative analysis of methylated alleles (QAMA), enzymatic regional methylation assay (ERMA), QBSUPT, MethylQuant, Quantitative PCR sequencing and oligonucleotide-based microarray systems, Pyrosequencing, Meth-DOP-PCR. A review of some useful techniques is provided in Nucleic acids research, 1998, Vol. 26, No. 10, 2255-2264, Nature Reviews, 2003, Vol. 3, 253-266; Oral Oncology, 2006, Vol. 42, 5-13, which references are incorporated herein in their entirety. Any of these techniques may be utilised in accordance with the present invention, as appropriate.

Additional techniques useful in the methods of the invention include those which utilize the ability of the methyl binding domain (MBD) of the MeCP2 protein to selectively bind to methylated DNA sequences (Cross et al, 1994; Shiraishi et al, 1999). Alternatively, the MBD may be obtained from MBP, MBP2, MBP4 or poly-MBD (Jorgensen et al., 2006). In one method, restriction exonuclease digested genomic DNA is loaded onto expressed His-tagged methyl-CpG binding domain that is immobilized to a solid matrix and used for preparative column chromatography to isolate highly methylated DNA sequences. Such methylated DNA enrichment-step may supplement the methods of the invention. Several other methods for detecting methylated CpG islands are well known in the art and include amongst others methylated-CpG island recovery assay (MIRA). Any of these methods may be employed in the present invention where desired.

In one specific embodiment, the methylation status of the HPV genome is determined using methylation specific PCR (MSP), or an equivalent amplification technique. The MSP technique will be familiar to one of skill in the art. In the MSP approach, DNA may be amplified using primer pairs designed to distinguish methylated from unmethylated DNA by taking advantage of sequence differences as a result of sodium-bisulphite treatment (Herman et al., 1996; and WO 97/46705).

A specific example of the MSP technique is designated real-time quantitative MSP (QMSP), which permits reliable quantification of methylated DNA in real time. These methods are generally based on the continuous optical monitoring of an amplification procedure and utilise fluorescently labelled reagents whose incorporation in a product can be quantified and whose quantification is indicative of copy number of that sequence in the template. One such reagent is a fluorescent dye, called SYBR Green I that preferentially binds double-stranded DNA and whose fluorescence is greatly enhanced by binding of double-stranded DNA. Alternatively, labelled primers and/or labelled probes can be used. They represent a specific application of the well known and commercially available real-time amplification techniques such as hydrolytic probes (TAQMAN®), hairpin probes (MOLECULAR BEACONS®), hairpin primers (AMPLIFLUOR®), hairpin probes integrated into primers (SCORPION®), oligonucleotide blockers and primers incorporating complementary sequences of DNAzymes (DzyNA®), specific interaction between two modified nucleotides (Plexor™) etc as described in more detail herein. Often, these real-time methods are used with the polymerase chain reaction (PCR).

An alternative to MSP is the so-called HeavyMethyl technique. This method is described in detail in WO 02/072880 for example. In this method, the primers used in the amplification do not need to be methylation specific (although they can also serve this function if desired). Instead, non-extendible oligonucleotide blockers provide for the ability to discriminate between methylated and unmethylated DNA following bisulphite treatment instead of the primers themselves. The blockers bind to bisulphite-treated DNA in a methylation-specific manner, and their binding sites may optionally overlap the primer binding sites. When the blocker is bound, the primer cannot bind and/or direct complete amplification and therefore an amplification product is not generated. The HeavyMethyl technique can be used in combination with real-time or end-point detection, as required, in the methods of the invention.

Thus, in one embodiment, the methylation status of the HPV genome is determined by methylation specific PCR/amplification and/or by HeavyMethyl, which may be a real-time or end point version thereof. In specific embodiments, the real time PCR/amplification involves use of hairpin primers (Amplifluor)/hairpin probes (Molecular Beacons)/hydrolytic probes (Taqman)/FRET probe pairs (Lightcycler)/primers incorporating a hairpin probe (Scorpion)/primers incorporating complementary sequences of DNAzymes that cleave a reporter substrate included in the reaction mixture (DzyNA®)/fluorescent dyes (SYBR Green etc.)/oligonucleotide blockers/the specific interaction between two modified nucleotides (Plexor). For Scorpion type primers, the primer and/or the probe may distinguish between methylated and unmethylated DNA following bisulphite treatment as required.

Real-Time PCR detects the accumulation of amplicon during the reaction. Real-time methods do not need to be utilised, however. Many applications do not require quantification and real-time PCR is used principally as a tool to obtain convenient results presentation and storage, and at the same time to avoid post-PCR handling. Analyses can be performed only to determine whether the target DNA is present in the sample or not. End point verification is carried out after the amplification reaction has finished. This knowledge can be used in a medical diagnostic laboratory for example, in the methods of the invention. In the majority of such cases, the quantification of DNA template is not very important. Amplification products may simply be run on a suitable gel, such as an agarose gel, to determine if the expected sized products are present. This may involve use of ethidium bromide staining and visualisation of the DNA bands under a UV illuminator for example. Alternatively, fluorescence or energy transfer can be measured to determine the presence of the methylated DNA. The end-point PCR fluorescence detection technique can use the same approaches as widely used for Real Time PCR: examples include the TaqMan assay, Molecular Beacons, Scorpion, Amplifluor etc as discussed in detail above. As an example, <<Gene>> detector allows the measurement of fluorescence directly in PCR tubes.

In real-time embodiments, quantitation may be on an absolute basis, or may be relative to a constitutively methylated DNA standard, or may be relative to an unmethylated DNA standard. Methylation status may be determined by using the ratio between the signal of the marker under investigation and the signal of a reference gene where methylation status is known (such as β-actin for example), or by using the ratio between the methylated marker and the sum of the methylated and the non-methylated marker. Alternatively, absolute copy number of the methylated marker gene can be determined. Suitable reference genes for the present invention include beta-actin, glyceraldehyde-3-phosphate dehydrogenase (GAPDH), ribosomal RNA genes such as 18S ribosomal RNA and RNA polymerase II gene (Radonic A. et al., Biochem Biophys Res Commun. 2004 Jan. 23; 313(4):856-62).

In one embodiment, each clinical sample is measured in duplicate and for both Ct values (cycles at which the amplification curves crossed the threshold value, set automatically by the relevant software) copy numbers are calculated. The average of both copy numbers (for each gene) is used for the result classification. To quantify the final results for each sample two standard curves are used, one for either the reference gene (β-actin or the non-methylated marker) and one for the methylated version of the marker. The results of all clinical samples (when m-Gene was detectable) are expressed as 1000 times the ratio of “copies m-Gene”/“copies β-actin” or “copies m-Gene”/“copies u-Gene+m-Gene” and then classified accordingly (methylated, non-methylated or invalid) (u=unmethylated; m=methylated).

In one embodiment, primers useful in determining the methylation status of the HPV genome (by MSP) are provided. These primers comprise, consist essentially of or consist of the nucleotide sequences set forth as follows:

| | |
|---|---|
| MSP-hpv16-Methylated Sense | |
| TGCGATATAAACGTTTTGTAAAAC | (SEQ ID NO: 43) |
| MSP-hpv16-Methylated Antisense | |
| AATATACCCAATACGTCCGC | (SEQ ID NO: 44) |
| MSP-hpv16-Unmethylated Sense | |
| TAATGTGATATAAATGTTTTGTAAAAT | (SEQ ID NO: 45) |
| MSP-hpv16-Unmethylated Antisense | |
| AAATATACCCAATACATCCACCT | (SEQ ID NO: 46) |

These primers are useful in determining the methylation status of the HPV-16 L2 gene and, in addition to being useful in the methods of the invention, form separate aspects of the invention. The invention thus provides a primer for use in methylation specific PCR (MSP) to determine the methylation status of a HPV-16 genome selected from the primers comprising the nucleotide sequences set forth as SEQ ID NO: 42 to 46 and functional derivatives thereof which retain functionality in MSP. Effectively, the methods of the invention may involve determining the methylation status in regions of the HPV16 genome defined by the sequence between (and including) the primer binding sites. Further characteristics of these primers are summarized in the detailed description (experimental part) below. Thus, the methods of the invention may comprise, consist essentially of or consist of determination of the methylation status of a plurality, up to all (110 CpG residues for the HPV16 genome) in individual increments, CpG residues in the nucleotide sequences which can be amplified using the primers of table 2 and SEQ ID NOs 43 to 46, as defined herein. It is noted that variants of these sequences may be utilised in the present invention. In particular, additional sequence specific flanking sequences may be added, for example to improve binding specificity, as required. Variant sequences preferably have at least 90%, at least 91%, at least 92%, at least 93%, at least 94%, at least 95%, at least 96%, at least 97%, at least 98%, or at least 99% nucleotide sequence identity with the nucleotide sequences of the primers set forth above (and in table 2). The primers may incorporate synthetic nucleotide analogues as appropriate or may be RNA or PNA based for example, or mixtures thereof. Primers may be labelled with suitable probes or combined with probes to allow real-time and/or end point detection. Such probes, and in certain embodiments the primers themselves, may be fluorescently labelled to facilitate detection. A range of alternative fluorescent donor and acceptor moieties/FRET pairs may be utilised as appropriate. In addition to being labelled with the fluorescent donor and acceptor moieties, the primers (or probes as appropriate) may include modified oligonucleotides and other appending groups and labels provided that the functionality as a primer in the methods of the invention is not compromised. Similarly alternative fluorescent donor and acceptor moieties/FRET pairs may be utilised as appropriate. Molecules that are commonly used in FRET include fluorescein, 5-carboxyfluorescein (FAM), 2′7′-dimethoxy-4′5′-dichloro-6-carboxyfluorescein (JOE), rhodamine, 6-carboxyrhodamine (R6G), N,N,N′,N′-tetramethyl-6-carboxyrhodamine (TAMRA), 6-carboxy-X-rhodamine (ROX), 4-(4′-dimethylaminophenylazo)benzoic acid (DABCYL), and 5-(2′-aminoethyl)aminonaphthalene-1-sulfonic acid (EDANS). Whether a fluorophore is a donor or an acceptor is defined by its excitation and emission spectra, and the fluorophore with which it is paired. For example, FAM is most efficiently excited by light with a wavelength of 488 nm, and emits light with a spectrum of 500 to 650 nm, and an emission maximum of 525 nm. FAM is a suitable donor fluorophore for use with JOE, TAMRA, and ROX (all of which have their excitation maximum at 514 nm).

Thus, in one embodiment, a donor moiety and acceptor moiety are selected from 5-carboxyfluorescein (FAM), 2′7′-dimethoxy-4′5′-dichloro-6-carboxyfluorescein (JOE), rhodamine, 6-carboxyrhodamine (R6G), N,N,N′-tetramethyl-6-carboxyrhodamine (TAMRA), 6-carboxy-X-rhodamine (ROX), 5-(2′-aminoethyl)aminonapthalene-1-sulfonic acid (EDANS), anthranilamide, coumarin, terbium chelate derivatives, Malachite green, Reactive Red 4, DABCYL, tetramethyl rhodamine, pyrene butyrate, eosine nitrotyrosine, ethidium, and Texas Red. In a further embodiment, a donor moiety is selected from fluorescein, 5-carboxyfluorescein (FAM), rhodamine, 5-(2′-aminoethyl)aminonapthalene-1-sulfonic acid (EDANS), anthranilamide, coumarin, terbium chelate derivatives, Malachite green, and Reactive Red 4, and an acceptor moiety is selected from DABCYL, rhodamine, tetramethyl rhodamine, pyrene butyrate, eosine nitrotyrosine, ethidium, and Texas Red. In specific embodiments, the donor moiety is fluorescein or a derivative thereof, and the acceptor moiety is DABCYL. The fluorescein derivative may comprise, consist essentially of or consist of 6-carboxy fluorescein.

For all aspects and embodiments of the invention, the primers and/or probes where utilised, may be labelled with donor and acceptor moieties as required during chemical synthesis of the primers or the label may be attached following synthesis using any suitable method. Many such methods are available and well characterised in the art.

In a further embodiment of the methods of the invention, bisulphite sequencing is utilised in order to determine the methylation status of the HPV genome. Bisulphite sequencing may be particularly useful in embodiments where the methylation status of the whole HPV genome is determined. By “whole HPV genome” is meant determination of the methylation status of selected CpG residues throughout the HPV genome. This may be carried out at regular intervals. Suitable residues to investigate may be determined by known methods such as through use of computer software to predict and identify CpG islands. This “global” approach provides particularly sensitive results. The HPV-16 methylome is shown in FIG. 3 and indicates the global approach which may be utilised. Sequencing primers may be designed for use in sequencing through the important CpG islands in the HPV genome. Thus, primers may be designed in both the sense and antisense orientation to direct sequencing across the relevant regions of the HPV genome.

Suitable primers for use in the methods of the invention are set forth as SEQ ID NO: 1 to 42 (and in table 1). Thus, the invention provides a primer for use in bisulphite sequencing of a HPV genome selected from the primers comprising the nucleotide sequences set forth as SEQ ID NO: 1 to 42 and functional derivatives thereof which retain functionality in bisulphite sequencing. All appropriate combinations of these primers may be utilised as required. These primers are useful in determining the methylation status of the HPV-16 genome and, in addition to being useful in the methods of the invention, form separate aspects of the invention. Effectively, the methods of the invention may involve determining the methylation status in regions of the HPV16 genome defined by the sequence between (and including) the primer binding sites. Thus, the methods of the invention may involve determination of the methylation status of a plurality, up to all, CpG residues in the nucleotide sequences which can be sequenced using the primers of table 1 and SEQ ID NOs 1 to 42, as defined herein. Further characteristics of these primers are summarized in the detailed description (experimental part) below. It is noted that variants of these sequences may be utilised in the present invention. In particular, additional sequence specific flanking sequences may be added, for example to improve binding specificity, as required. Variant sequences preferably have at least 90%, at least 91%, at least 92%, at least 93%, at least 94%, at least 95%, at least 96%, at least 97%, at least 98%, or at least 99% nucleotide sequence identity with the nucleotide sequences of the primers set forth above (and in table 1). The primers may incorporate synthetic nucleotide analogues as appropriate or may be RNA or PNA based for example, or mixtures thereof. Primers may be fluorescently labelled to facilitate detection.

Other nucleic acid amplification techniques, in addition to PCR (which includes real-time versions thereof and variants such as nested PCR), may also be utilised in the methods of the invention, as appropriate, to detect the methylation status of the HPV genome. Such amplification techniques are well known in the art, and include methods such as NASBA (Compton, 1991), 3SR (Fahy et al., 1991) and Transcription Mediated Amplification (TMA). Other suitable amplification methods include the ligase chain reaction (LCR) (Barringer et al, 1990), selective amplification of target polynucleotide sequences (U.S. Pat. No. 6,410,276), consensus sequence primed polymerase chain reaction (U.S. Pat. No 4,437,975), arbitrarily primed polymerase chain reaction (WO 90/06995), invader technology, strand displacement technology, and nick displacement amplification (WO 2004/067726). This list is not intended to be exhaustive; any nucleic acid amplification technique may be used provided the appropriate nucleic acid product is specifically amplified. Thus, these amplification techniques may be tied in to MSP and/or HeavyMethyl and/or bisulphite sequencing techniques for example.

As discussed above, sequence variation that reflects the methylation status at CpG dinucleotides in the original genomic DNA offers different approaches to primer design. Both primer types may be utilised in the methods of the invention. Firstly, primers may be designed that themselves do not cover any potential sites of DNA methylation. Sequence variations at sites of differential methylation are located between the two primers. Such primers are used in bisulphite genomic sequencing, COBRA and Ms-SnuPE for example. Secondly, primers may be designed that anneal specifically with either the methylated or unmethylated version of the converted sequence. If there is a sufficient region of complementarity, e.g., 12, 15, 18, or 20 nucleotides, to the target, then the primer may also contain additional nucleotide residues that do not interfere with hybridization but may be useful for other manipulations. Examples of such other residues may be sites for restriction endonuclease cleavage, for ligand binding or for factor binding or linkers or repeats. The oligonucleotide primers may or may not be such that they are specific for modified methylated residues.

One way to distinguish between modified and unmodified DNA is to hybridize oligonucleotide primers which specifically bind to one form or the other of the DNA. After hybridization, an amplification reaction can be performed and amplification products assayed. The presence of an amplification product indicates that a sample hybridized to the primer. The specificity of the primer indicates whether the DNA had been modified or not, which in turn indicates whether the DNA had been methylated or not.

Another way to distinguish between modified and unmodified DNA is to use oligonucleotide probes which may also be specific for certain products. Such probes may be hybridized directly to modified DNA or to amplification products of modified DNA. Oligonucleotide probes can be labelled using any detection system known in the art. These include but are not limited to fluorescent moieties, radioisotope labelled moieties, bioluminescent moieties, luminescent moieties, chemiluminescent moieties, enzymes, substrates, receptors, or ligands.

As discussed above, in the MSP technique, amplification is achieved with the use of primers specific for the sequence of the gene whose methylation status is to be assessed. In order to provide specificity for the nucleic acid molecules, primer binding sites corresponding to a suitable region of the sequence may be selected. The skilled reader will appreciate that the nucleic acid molecules may also include sequences other than primer binding sites which are required for detection of the methylation status of the gene, for example RNA Polymerase binding sites or promoter sequences may be required for isothermal amplification technologies, such as NASBA, 3SR and TMA.

When determining methylation status, it may be beneficial to include suitable controls. This may be particularly useful in applications of the methods of the invention in which staging or progression of a particular disease is the aim. Suitable controls may also be advantageous in order to ensure the methods of the invention are working correctly and reliably. Thus, in one embodiment the determined methylation status (of the HPV genome) is compared to a control. In one embodiment the control is a sample taken from the same subject at an earlier time point. This allows monitoring of disease progression in a subject. It may also allow the effectiveness of a given treatment to be determined—if the methylation status does not progress to a more methylated level, or in fact decreases, it may be determined that the treatment is having the desired effect. Thus, the basic correlation of the invention is that an increased level of methylation of the HPV genome (up to and including the entire methylome) indicates the progression of the disease to a more advanced form.

Additionally or alternatively, the control may comprise, consist essentially of or consist of one or more reference samples representing the methylation status of the HPV genome at a defined stage of the disease. Thus, a whole collection of suitable control samples taken from subjects at various stages of the disease may be utilised as a reference point. Note, the samples themselves need not be available when the methods of the invention are carried out. The results of determining the methylation status of the HPV genome are all that is required to compare with the output from the methods of the invention. Thus, it can be readily envisaged that a large databank of information can be accumulated and used as a point of reference to facilitate interpretation of results achieved using the methods of the invention. As discussed above, in one embodiment the methylation patterns presented in FIG. 3 are utilised as a reference in order to facilitate the diagnosis.

Other suitable controls may include assessing the methylation status of a gene known to be methylated in the sample. This experiment acts as a positive control to ensure that false negative results are not obtained (i.e. a conclusion of a lack of methylation is made even though the HPV genome may, in fact, be methylated). The gene may be one which is known to be methylated in the sample under investigation or it may have been artificially methylated, for example by using a suitable methyltransferase enzyme, such as Sssl methyltransferase.

Additionally or alternatively, suitable negative controls may be employed with the methods of the invention. Here, suitable controls may include assessing the methylation status of a gene known to be unmethylated or carrying out an amplification in the absence of DNA (for example by using a water only sample). This experiment acts as a negative control to ensure that false positive results are not obtained (i.e. a conclusion of methylation is made even though the HPV genome may, in fact, be unmethylated). The gene may be one which is known to be unmethylated in the sample under investigation or it may have been artificially demethylated, for example by using a suitable DNA methyltransferase inhibitor.

As mentioned above, the basic correlation of the invention is that an increased level of methylation of the HPV genome indicates the progression of the disease to a more advanced form. Accordingly, the invention provides a method of monitoring the progression of a disease caused by a human papillomavirus (HPV) infection in response to a treatment directed against the disease in a test sample obtained from a subject comprising determining the methylation status of a HPV genome before and following treatment of the subject wherein the presence of decreased methylation of the HPV genome following treatment indicates a positive effect of the treatment on the disease in terms of successfully inhibiting disease progression. The presence of equal methylation before and following treatment may indicate that the treatment has been successful in preventing progression of the disease. The presence of increased methylation following treatment may indicate that the treatment has been unsuccessful in preventing progression of the disease (and that therefore alternative treatments should be explored). For the avoidance of doubt, it is hereby stated that all embodiments and applications of the (other) methods of the invention apply mutatis mutandis to this particular aspect of the invention and are not repeated for reasons of conciseness.

The application of the methods of the present invention to extremely small amounts of abnormally-methylated DNA, that are released into test samples comprised of fluids may require the generation and amplification of a DNA library before testing for methylation of any specific gene. Suitable methods on whole genome amplification and libraries generation for such amplification (e.g. Methylplex and Enzyplex technology, Rubicon Genomics) are described in US2003/0143599, WO2004/081225 and WO2004/081183 for example. In addition, WO2005/090507 describes library generation/amplification methods that require either bisulfite conversion or non-bisulfite based application. Bisulfite treatment may occur before or after library construction and may require the use of adaptors resistant to bisulfite conversion. Meth-DOP-PCR (Di Vinci et al, 2006), a modified degenerate oligonucleotide-primed PCR amplification (DOP-PCR) that is combined with MSP, provides another suitable method for specific detection of methylation in small amounts of DNA. Improved management of patient care may require these existing methods and techniques to supplement the methods of the invention.

As mentioned above, determining the methylation status at a number of locations in the HPV genome may be advantageous. Thus, in one embodiment, the methods of the invention are utilised in order to determine the methylation status of at least the E7/E1/E2/E4/E5/L2/L1 gene. In one embodiment, the methylation status of the entire HPV-16 genome is determined. Moreover, in order to improve the sensitivity of the methods of the invention the methods may comprise detecting an epigenetic change in a panel of genes comprising at least two, three, four, five, six etc. up to all of the genes, and optionally also including or selected from additional regions such as the Upstream Regulatory Region (URR). The level of methylation at each point of investigation contributes to the output of the methods of the invention in terms of diagnosing, staging or monitoring the progression of the disease. Thus, the methods of the invention generally include investigation of a significant number of cytosine nucleotides (in the context of CpG dinucleotide pairs) within the HPV genome. As aforementioned, the methods of the invention may involve determination of the methylation status of a plurality, up to all, CpG residues in the nucleotide sequences which can be sequenced or amplified using the primers of tables 1 and 2 and SEQ ID NOs 1 to 46, as defined herein. Even within each of the genes or regions of the genome, as discussed herein, which may be investigated, it is possible to investigate the methylation status of multiple cytosine residues. Individual primers and probes used in the methods of the invention may investigate the methylation status of a plurality of cytosine residues in each case through appropriate primer and probe design, as would be immediately apparent to the skilled person. For example, the primers and probes may be designed to overlap 1, 2, 3, 4, 5, 6, 7, 8, 9, 10 etc cytosine residues and thus investigate the methylation status of multiple sites in a single reaction.

Since DNA methylation, particularly in the promoter or other regulatory regions of a gene generally corresponds to a decrease in expression of the corresponding gene, the methods of the invention may employ determination of gene expression levels as an indication of the methylation status of the HPV genome. As the disease progresses and methylation levels increase gene expression may decrease in corresponding fashion. Thus, the invention provides a method of diagnosing and/or monitoring the progression of or otherwise staging a disease caused by a human papillomavirus (HPV) infection in a test sample obtained from a subject comprising determining the methylation status of a HPV genome wherein the presence of hypermethylation of the HPV genome indicates a positive diagnosis of the disease and/or an increased level of methylation of the HPV genome indicates the progression of the disease to a more advanced form and wherein levels of expression of one or more. HPV genes are measured as an indication of the methylation status of the HPV genome.

In a specific embodiment, the methods of the invention include determining the methylation status of the HPV genome by determining whether E6 and/or E7 is overexpressed. Such overexpression may be considered surprising in light of the general correlation between methylation and decreased gene expression. However, as discussed in detail herein, methylation of E2 binding sites in the Upstream Regulatory Region (URR) of the HPV(16) genome are associated with high levels of expression of E6 and E7 oncoproteins. Overexpression of E6 and E7 appears to be due to the fact that the E2 viral protein cannot bind to methylated E2-binding sites. This also indicates that determining the methylation status of the E2-binding sites in the URR of the HPV(16) genome is useful in the methods of the invention. Likewise, methods for determining whether E2 has bound to its binding sites within the URR may also be employed in the methods of the invention as an indication of the methylation status of the HPV genome. Any suitable method of determining protein binding may be employed. In a specific embodiment, chromatin immunoprecipitation techniques are used to investigate whether the E2 protein has bound and thus whether the E2-binding sites are methylated. Accordingly, the invention provides a method of diagnosing and/or monitoring the progression of or otherwise staging a disease caused by a human papillomavirus (HPV) infection in a test sample obtained from a subject comprising determining the expression levels of E6 and/or E7 wherein the presence of overexpression of E6 and/or E7 indicates a positive diagnosis of the disease and/or an increased level of expression of E6 and/or E7 indicates the progression of the disease to a more advanced form.

Gene expression may be determined at the RNA or protein level, as would be readily appreciated by one skilled in the art. “Overexpression” indicates an increase in expression relative to the level of expression in which there is no or normal levels of methylation of the HPV genome, in particular at the E2 binding sites in the URR. With respect to E6 and/or E7, overexpression means increased expression as compared to the level of expression when the E2-binding sites in the URR are unmethylated. Overexpression may be determined by assessing protein activity, rather than by directly looking at expression levels themselves. Changes in the level of expression may, as necessary, be measured in order to determine if it is statistically significant in the sample. This helps to provide a reliable test for the methods of the invention. Any method for determining whether the expression levels are significantly altered may be utilised. Such methods are well known in the art and routinely employed. For example, statistical analyses may be performed. One example involves an analysis of variance test. Typical P values for use in such a method would be P values of <0.05 or 0.01 or 0.001 when determining whether the relative expression or activity is statistically significant. A change in expression may be deemed significant if there is at least a 10% change for example. The test may be made more selective by making the change at least 15%, 20%, 25%, 30%, 35%, 40% or 50%, for example, in order to be considered statistically significant.

As discussed in respect of the methods involving direct determination of methylation levels in the HPV genome, levels of expression or activity may be determined with reference to a control sample. Thus, for example, expression of E6 and/or E7 in the test sample may be measured and compared to control samples in which expression levels of E6 and/or E7 were determined and where there was no, partial or complete methylation of the E2-binding sites respectively.

Suitable additional controls may also be included to ensure that the test is working properly, such as measuring levels of expression or activity of a suitable reference gene in both test and control samples. Suitable reference genes for the present invention include beta-actin, glyceraldehyde-3-phosphate dehydrogenase (GAPDH), ribosomal RNA genes such as 18S ribosomal RNA and RNA polymerase II gene (Radonic A. et al., Biochem Biophys Res Commun. 2004 Jan 23; 313(4):856-62).

Expression of a nucleic acid can be measured at the RNA level or at the protein level. Cells in test samples can be lysed and the mRNA levels in the lysates, or in the RNA purified or semi-purified from the lysates, determined. Alternatively, methods can be used on un-lysed tissues or cell suspensions. Suitable methods for determining expression at the RNA level are well known in the art and described herein.

Methods employing nucleic acid probe hybridization to the relevant transcript(s), such as the E6 and/or E7 transcripts may be employed for measuring the presence and/or level of the respective mRNA. Such methods are well known in the art and include use of nucleic acid probe arrays (microarray technology) and Northern blots. Advances in genomic technologies now permit the simultaneous analysis of thousands of genes, although many are based on the same concept of specific probe-target hybridization. Sequencing-based methods are an alternative. These methods started with the use of expressed sequence tags (ESTs), and now include methods based on short tags, such as serial analysis of gene expression (SAGE) and massively parallel signature sequencing (MPSS). Differential display techniques provide yet another means of analyzing gene expression; this family of techniques is based on random amplification of cDNA fragments generated by restriction digestion, and bands that differ between two tissues identify cDNAs of interest.

In one embodiment, the levels of gene expression are determined using reverse transcriptase polymerase chain reaction (RT-PCR). RT-PCR is a well known technique in the art which relies upon the enzyme reverse transcriptase to reverse transcribe mRNA to form cDNA, which can then be amplified in a standard PCR reaction. Protocols and kits for carrying out RT-PCR are extremely well known to those of skill in the art and are commercially available.

The RT-PCR can be carried out in a non-quantitative manner. End-point RT-PCR measures changes in expression levels using three different methods: relative, competitive and comparative. These traditional methods are well known in the art. Alternatively, RT-PCR is carried out in a real time and/or in a quantitative manner. Real time quantitative RT-PCR has been thoroughly described in the literature (see Gibson et al for an early example of the technique) and a variety of techniques are possible. Examples include use of hydrolytic probes (Taqman), hairpin probes (Molecular Beacons), FRET probe pairs (LightCycler (Roche)), hairpin probes attached to primers (Scorpion), hairpin primers (Plexor and Amplifluor), DzyNA and oligonucleotide blocker systems. All of these systems are commercially available and well characterised, and may allow multiplexing (that is, the determination of expression of multiple genes in a single sample).

TAQMAN was one of the earliest available real-time PCR techniques and relies upon a probe which binds between the upstream and downstream primer binding sites in a PCR reaction. A TAQMAN probe contains a 5′ fluorophore and a 3′ quencher moiety. Thus, when bound to its binding site on the DNA the probe does not fluoresce due to the presence of the quencher in close proximity to the fluorophore. During amplification, the 5′-3′ exonuclease activity of a suitable polymerase such as Taq digests the probe if it is bound to the strand being amplified. This digestion of the probe causes displacement of the fluorophore. Release of the fluorophore means that it is no longer in close proximity to the quencher moiety and this therefore allows the fluorophore to fluoresce. The resulting fluorescence may be measured and is in direct proportion to the amount of target sequence that is being amplified. These probes are sometimes generically referred to as hydrolytic probes.

In the Molecular Beacons system, the probe is again designed to bind between the primer binding sites. However, here the probe is a hairpin shaped probe. The hairpin in the probe when not bound to its target sequence means that a fluorophore attached to one end of the probe and a quencher attached to the other end of the probe are brought into close proximity and therefore internal quenching occurs. Only when the target sequence for the probe is formed during the PCR amplification does the probe unfold and bind to this sequence. The loop portion of the probe acts as the probe itself, while the stem is formed by complimentary arm sequences (to respective ends of which are attached the fluorophore and quencher moiety). When the beacon probe detects its target, it undergoes a conformational change forcing the stem apart and this separates the fluorophore and quencher. This causes the energy transfer to the quencher to be disrupted and therefore restores fluorescence.

During the denaturation step, the Molecular Beacons assume a random-coil configuration and fluoresce. As the temperature is lowered to allow annealing of the primers, stem hybrids form rapidly, preventing fluorescence. However, at the annealing temperature, Molecular Beacons also bind to the amplicons, undergo conformational reorganisation, leading to fluorescence. When the temperature is raised to allow primer extension, the Molecular Beacons dissociate from their targets and do not interfere with polymerisation. A new hybridisation takes place in the annealing step of every cycle, and the intensity of the resulting fluorescence indicates the amount of accumulated amplicon.

Scorpions primers are based upon the same principles as Molecular Beacons. However, here, the probe is bound to, and forms an integral part of, an amplification primer. The probe has a blocking group at its 5′ end to prevent amplification through the probe sequence. After one round of amplification has been directed by this primer, the target sequence for the probe is produced and to this the probe binds. Thus, the name “scorpion” arises from the fact that the probe as part of an amplification product internally hybridises to its target sequence thus forming a tail type structure. Probe-target binding is kinetically favoured over intrastrand secondary structures. Scorpions primers were first described in the paper “Detection of PCR products using self-probing amplicons and fluorescence” (Nature Biotechnology. 17, p 804-807 (1999)).

In similar fashion to Scorpions primers, Amplifluor primers rely upon incorporation of a Molecular Beacon type probe into a primer. Again, the hairpin structure of the probe forms part of an amplification primer itself. However, in contrast to Scorpions type primers, there is no block at the 5′ end of the probe in order to prevent it being amplified and forming part of an amplification product. Accordingly, the primer binds to a template strand and directs synthesis of the complementary strand. The primer therefore becomes part of the amplification product in the first round of amplification. When the complimentary strand is synthesised amplification occurs through the hairpin structure. This separates the fluorophore and quencher molecules, thus leading to generation of fluorescence as amplification proceeds.

DzyNA primers incorporate the complementary/antisense sequence of a 10-23 nucleotide DNAzyme. During amplification, amplicons are produced that contain active (sense) copies of DNAzymes that cleave a reporter substrate included in the reaction mixture. The accumulation of amplicons during PCR/amplification can be monitored in real time by changes in fluorescence produced by separation of fluorophore and quencher dye molecules incorporated into opposite sides of a DNAzyme cleavage site within the reporter substrate. The DNAzyme and reporter substrate sequences can be generic and hence can be adapted for use with primer sets targeting various genes or transcripts (Todd et al., Clinical Chemistry 46:5, 625-630 (2000)).

The Plexor™ qPCR and qRT-PCR Systems take advantage of the specific interaction between two modified nucleotides to achieve quantitative PCR analysis. One of the PCR primers contains a fluorescent label adjacent to an iso-dC residue at the 5′ terminus. The second PCR primer is unlabeled. The reaction mix includes deoxynucleotides and iso-dGTP modified with the quencher dabcyl. Dabcyl-iso-dGTP is preferentially incorporated at the position complementary to the iso-dC residue. The incorporation of the dabcyl-iso-dGTP at this position results in quenching of the fluorescent dye on the complementary strand and a reduction in fluorescence, which allows quantitation during amplification. For these multiplex reactions, a primer pair with a different fluorophore is used for each target sequence.

Real time quantitative techniques produce a fluorescent read-out that can be continuously monitored. Fluorescence signals are generated by dyes that are specific to double stranded DNA, like SYBR Green, or by sequence-specific fluorescently-labeled oligonucleotide primers or probes. Each of the primers or probes can be labelled with a different fluorophore to allow specific detection. These real time quantitative techniques are advantageous because they keep the reaction in a “single tube”. This means there is no need for downstream analysis in order to obtain results, leading to more rapidly obtained results. Furthermore, keeping the reaction in a “single tube” environment reduces the risk of cross contamination and allows a quantitative output from the methods of the invention. This may be particularly important in a clinical setting for the present invention.

It should be noted that whilst PCR is a preferred amplification method, to include variants on the basic technique such as nested PCR, equivalents may also be included within the scope of the invention. Examples include without limitation isothermal amplification techniques such as NASBA, 3SR, TMA and triamplification, all of which are well known in the art and commercially available. Other suitable amplification methods without limitation include the ligase chain reaction (LCR) (Barringer et al, 1990), MLPA, selective amplification of target polynucleotide sequences (U.S. Pat. No. 6,410,276), consensus sequence primed polymerase chain reaction (U.S. Pat. No. 4,437,975), invader technology (Third Wave Technologies, Madison, Wis.), strand displacement technology, arbitrarily primed polymerase chain reaction (WO90/06995) and nick displacement amplification (WO2004/067726).

Suitable methods for determining expression, for example of E6 and/or E7, at the protein level are also well known to one of skill in the art. Examples include western blots, immunohistochemical staining and immunolocalization, immunofluorescence, enzyme-linked immunosorbent assay (ELISA), immunoprecipitation assays, complement fixation assay, agglutination reactions, radioimmunoassay, flow cytometry, mass spectrophotometry, and equilibrium dialysis. These methods generally depend upon a reagent specific for identification of the appropriate gene product. Any suitable reagent may be utilised such as lectins, receptors, nucleic acids, antibodies etc. The reagent is preferably an antibody and may comprise monoclonal or polyclonal antibodies. Fragments and derivatized antibodies may also be utilised, to include without limitation Fab fragments, ScFv, single domain antibodies, nano-antibodies, heavy chain antibodies, aptamers etc. which retain gene product binding function. Any detection method may be employed in accordance with the invention. The nature of the reagent is not limited except that it must be capable of specifically identifying the appropriate gene product.

Measurement of expression of a gene or protein on its own does not necessarily conclusively indicate that the silencing is epigenetic, as the mechanism of silencing could be genetic, for example, by somatic mutation. Accordingly, in one embodiment, the methods of the invention incorporate an appropriate re-expression assay which is designed to reverse epigenetic silencing. Appropriate treatment of the sample using a demethylating agent, such as a DNA-methyltransferase (DMT) inhibitor may reverse epigenetic silencing of the relevant gene, or in the case of E6 and/or E7, E2-binding sites. Suitable reagents include, but are not limited to, DAC (5′-deazacytidine), TSA or any other treatment affecting epigenetic mechanisms present in cell lines. Typically, expression is reactivated or reversed upon treatment with such reagents, indicating that the silencing is epigenetic.

The methods of the invention may be applied to any HPV which contributes to a disease condition and wherein the progression of the disease is linked to increased methylation of the HPV genome. In a specific embodiment, the HPV is HPV16. HPV16 is a well known and characterised high risk HPV, which is specifically linked to the incidence of cervical cancer. The inventors have provided the entire methylome of HPV16 (110 methylation sites in 7904 nucleotides) for the first time herein.

The invention also provides kits which may be used in order to carry out the methods of the invention. The kits may incorporate any of the preferred features mentioned in connection with the various methods (and uses) of the invention herein. Accordingly, there is provided a kit for use in the methods of the invention comprising, consisting essentially of or consisting of a primer pair selected from the primers of SEQ ID NOs 1 to 46. These primers are described in greater detail herein. Suitable pairs are shown in tables 1 and 2 respectively. The kit may also comprise, consist essentially of or consist of a reagent for converting unmethylated cytosine residues to a changed nucleotide which displays different base pairing properties to cytosine, such as uracil but which does not convert methylated cytosine residues. The reagent may be a chemical or an enzyme for example. In one embodiment, the reagent comprises, consists essentially of or consists of a bisulphite reagent. In a more specific embodiment, the reagent comprises, consists essentially of or consists of sodium bisulphite. An enzyme such as a cytidine deaminase may be used in the methods and kits of the invention to mediate conversion as appropriate (e.g. see Bransteitter et al. PNAS USA (2003) Apr. 1; 100(7): 4102-4107).

In a further embodiment, the means for determining the methylation status of the HPV genome, that is to say the primers included in the kit, enable the detection to be carried out in a single reaction. Multiplexing is made possible for example through use of appropriate fluorophores having separable emission spectra. TaqMan probes, Molecular Beacons, Scorpions, etc., to include all suitable techniques and as discussed in greater detail above allow multiple markers to be measured in the same sample (multiplex PCR), since fluorescent dyes with different emission spectra may be attached to the different probes. Accordingly, suitably labelled probes and primers are encompassed by the kits of the invention.

Many suitable reagents for methylation detection are known in the art, and are discussed herein (which discussion applies here mutatis mutandis). In certain embodiments, the kit also includes means for carrying out the methylation detection in single tube format, which may optionally be in real time. Means for carrying out embodiments such as methylation specific PCR/amplification or HeavyMethyl in real time may comprise hairpin primers (Amplifluor), hairpin probes (Molecular Beacons), hydrolytic probes (Taqman), FRET probe pairs (Lightcycler), primers incorporating a hairpin probe (Scorpion), fluorescent dyes (SYBR Green etc.), DzyNA primers or oligonucleotide blockers. All appropriate combinations are envisaged by the invention.

The end-point PCR fluorescence detection technique can use the same approaches as widely used for Real Time PCR-TaqMan assay, Molecular Beacons, Scorpion etc. as discussed in greater detail herein. Accordingly, the kits of the invention may include means for carrying out the end-point methylation detection, such as methylation specific PCR or HeavyMethyl. The means for carrying out end-point methylation specific PCR/amplification may comprise primers and/or probes as explained for PCR in Real-time.

In the real-time and end-point detection embodiments, the probes for detection of amplification products may simply be used to monitor progress of the amplification reaction in real-time and/or they may also have a role in determining the methylation status of the HPV genome themselves. Thus, the probes may be designed in much the same fashion as the primers to take advantage of sequence differences following treatment with a suitable reagent such as sodium bisulphite dependent upon the methylation status of the appropriate cytosine residues (found in CpG dinucleotides).

The probes may comprise any suitable probe type for real-time detection of amplification products as discussed above. Notably, however, with the AMPLIFLUOR and SCORPION embodiments, the probes are an integral part of the primers which are utilised. The probes are typically fluorescently labelled, although other label types may be utilised as appropriate (such as mass labels or radioisotope labels). These probes are also suitable for end-point detection.

In one embodiment, the kit may also incorporate means for processing the test sample. Depending upon the sample to be analyzed this may include reagents such as a homogenization buffer. The means for processing a sample may further or alternatively comprise reagents for extraction/isolation/concentration/purification of DNA. Suitable reagents are known in the art and comprise, consist essentially of or consist of alcohols such as ethanol and isopropanol for precipitation of DNA. Salt-based precipitation may require high concentrations of salts to precipitate contaminants. The salt may comprise, consist essentially of or consist of potassium acetate and/or ammonium acetate for example. Organic solvents may also be included in the kits to extract contaminants from cell lysates. Thus, in one embodiment, the means for processing the sample comprise, consist essentially of or consist of phenol, chloroform and isoamyl alcohol to extract the DNA. Suitable combinations of reagents are envisaged as appropriate.

The kits may incorporate reagents for quantification of DNA such as those found in the Picogreen® dsDNA quantitation kit available from Molecular Probes, Invitrogen.

The kits may, in certain embodiments, also include means for obtaining a sample. For example, where the kits are used in the diagnosis, monitoring and staging of cervical cancer, the kits of the invention may also contain means for removing cervical cells from a patient for analysis. For example a spatula, such as an Ayre's spatula and/or a brush, such as an endocervical brush may be incorporated in the kits (or used in the methods) of the invention in order to obtain a cervical sample.

Sensitivity of detection, staging and monitoring may conceivably be improved by increasing the quantity of DNA in the sample. Accordingly, in one embodiment the means for processing a sample comprises, consists essentially of or consists of primers for directing amplification of HPV DNA in the sample. Any suitable primers which amplify the HPV genome, or relevant portions thereof, may be utilised. Preferably, the primers do not discriminate between methylated and unmethylated DNA (i.e. the primer binding sites lies outside of the CpG islands) thus providing a general increase in the amount of HPV DNA prior to determining whether the methylated form of the HPV genome is present in the sample. In a further embodiment, the primers are designed specifically for the HPV genome such that there is minimal sequence overlap, or sequence homology, with endogenous DNA from the subject under test (the infective HPV genome is considered exogenous to the genomic DNA, even if it has integrated). This means that the HPV genome will be selectively amplified, thus improving the sensitivity of the methods of the invention. The aim is to prevent non-specific amplification of genomic DNA from the subject which may influence the results.

Preferably, the homology is less than about 5%, less than about 10%, less than about 12.5%, less than about 15%, less than about 20%, less than about 30%, less than about 40%, 50%, 60%, 70% or 80% sequence identity with the corresponding nucleotide sequence from the genomic DNA of the subject under test. In one embodiment, there is no sequence identity with the corresponding nucleotide sequence from the DNA of the subject under test over approximately 10, 20, 30, 40 or 50 contiguous nucleotides. In another embodiment, there is less than about 10% or less than about 12.5%, 15%, 20%, 30%, 40%, 50% or 60% sequence identity over approximately 10, 20, 30, 40 or 50 contiguous nucleotides with the corresponding nucleotide sequence from the endogenous DNA of the subject under test.

As discussed with respect to the methods of the invention, suitable controls may be utilised in order to act as quality control for the methods. Accordingly, in one embodiment, the kit of the invention further comprises, consists essentially of or consists of one or more control nucleic acid molecules of which the methylation status is known. These (one or more) control nucleic acid molecules may include both nucleic acids which are known to be, or treated so as to be, methylated and/or nucleic acid molecules which are known to be, or treated so as to be, unmethylated. One example of a suitable internal reference gene, which is generally unmethylated, but may be treated so as to be methylated, is beta-actin.

Furthermore, the kit of the invention may further comprise, consist essentially of or consist of primers for the amplification of the control nucleic acid. These primers may be designed according to the control nucleic acid selected. Suitable probes and/or oligonucleotide blockers for use in determining the methylation status of the control nucleic acid molecules may also be incorporated into the kits of the invention. The probes may comprise any suitable probe type for real-time detection of amplification products. The discussion provided above applies mutatis mutandis.

The kits of the invention may additionally include suitable buffers and other reagents for carrying out the claimed methods of the invention. Thus, the discussion provided in respect of the methods of the invention as to the requirements for determination of the methylation status of the HPV genome apply mutatis mutandis here.

In one embodiment, the kit of the invention further comprises, consists essentially of, or consists of nucleic acid amplification buffers.

The kit may also additionally comprise, consist essentially of or consist of enzymes to catalyze nucleic acid amplification. Thus, the kit may also additionally comprise, consist essentially of or consist of a suitable polymerase for nucleic acid amplification. Examples include those from both family A and family B type polymerises, such as Taq, Pfu, Vent etc.

The various components of the kit may be packaged separately in separate compartments or may, for example be stored together where appropriate.

The kit may also incorporate suitable instructions for use, which may be printed on a separate sheet or incorporated into the kit packaging for example. For example, data may be provided to indicate typical levels of methylation which are expected for each stage of disease, based upon a number of previous results which have been accumulated and assessed. The results obtained using the kits may then be compared with this data to provide a diagnosis or indication of the stage or progression of the disease. The data may be provided in any suitable format—such as in software or printed form. For example, the methylation patterns presented in FIG. 3 may be presented and utilised as a reference in order to facilitate the diagnosis.

Other related methods and kits can be derived from the methylome information presented for the first time in the present invention. For each of these methods and kits, the embodiments and description above apply mutatis mutandis. Thus, the invention also provides a method of diagnosing cervical intraepithelial neoplasia in a test sample obtained from a subject comprising determining CpG dinucleotide loss in the L2 gene of a HPV-16 genome present in the sample wherein the loss of at least one CpG dinucleotide from the L2 gene is indicative of cervical intraepithelial neoplasia. In a specific embodiment, loss of two CpG dinucleotides from the L2 gene is indicative of cervical intraepithelial neoplasia. In order to facilitate these methods the position of the lost CpG dinucleotides indicated in FIG. 3 may be investigated, as discussed below. CpG dinucleotide loss may be determined by any suitable method. For example, sequencing may be utilised and in particular bisulphite sequencing may be used to identify loss of CpG dinucleotides from the HPV genome. The loss of the one or more CpG dinucleotides may be measured relative to the sequence of the L2 gene in a control. Suitable controls are discussed herein for other aspects of the invention. In one embodiment, the control is a wild type HPV-16 sample and/or an asymptomatic lesion. Thus, the sequence of the L2 gene can be readily determined with the known standard L2 gene sequence to determine whether CpG loss has occurred. A corresponding kit is also provided.

In a further related aspect, the invention also provides a method of diagnosing cervical intraepithelial neoplasia in a test sample obtained from a subject comprising determining the presence or absence of multiple mutations in a HPV-16 genome, wherein the presence of multiple mutations in the HPV-16 genome is indicative of the presence of cervical intraepithelial neoplasia. The mutations may be point mutations for example. In specific embodiments, the URR/E1/E2/E4/E5/L2/L1 regions/genes of the HPV genome are analysed for the (point) mutations. In a specific embodiment, the (point) mutations listed in FIG. 3 are investigated as an indicator of cervical intraepithelial neoplasia. The presence or absence of multiple (point) mutations may be determined by any suitable technique. Thus, sequencing, and in particular bisulphite sequencing may be employed in certain embodiments. Suitable controls are discussed herein for other aspects of the invention. In one embodiment, the control is a wild type HPV-16 sample and/or an asymptomatic lesion. Thus, the sequence of the L2 gene can be readily determined with the known standard L2 gene sequence to determine whether the point mutations are present. A corresponding kit is also provided.

The invention also provides for the use of the HPV16 DNA methylome in a number of diagnostic applications, including diagnosing and/or monitoring the progression of or otherwise staging a disease caused by a human papillomavirus (HPV) infection in a test sample obtained from a subject. Thus, the invention provides for the use of the HPV-16 DNA methylome as a reference for comparing obtained methylation patterns from a test sample. The information on the HPV methylome as presented in FIG. 3 may thus be employed as a reference. This comparison may be used in order to diagnose squamous cell carcinoma/cervical intraepithelial neoplasia.

### BRIEF DESCRIPTION OF THE FIGURES

FIG. 1.A. Papillomavirus life cycle; B. Progression from a benign cervical lesion to invasive cervical cancer. Adapted from Lowy, D. R. et al. (LSIL, low-grade squamous intraepithelial lesion; HSIL, high-grade squamous intraepithelial lesion; CIN, cervical intraepithelial neoplasia)

FIG. 2. Methylation status of a promoter CpG island of normal tissue and tumoral tissue. Adapted from Esteller, 2005. Grey boxes, exons; white circles, unmethylated CpGs; black circles, methylated CpGs.

FIG. 3. Methylation pattern of HPV16 genome using bisulphite sequencing.

FIG. 4. Expression results: Western blotting expression of HPV-16 E6 and E7 proteins in CaSki and SiHa cell lines.

FIG. 5. Methylation-Specific PCR (MSP) analyses of DNA methylation in the HPV-16 virus. “U” and “M” lanes indicates unmethylated and methylated sequences. Three CIN samples (smears 8, 9 and 10) demonstrate absence of HPV16 DNA methylation. In Vitro methylated DNA (IVD) is shown as positive control for methylated DNA sequences. Water is shown as negative PCR control.

FIG. 6. Results of Methylation Specific PCR carried out using a large collection (n=87) of human primary samples from the different stages of cervical carcinogenesis. MSP was carried out for the L2 region of the virus. The progressive presence of hypermethylation at the L2 locus in tumorigenesis is observed: none (0 of 10) in asymptomatic carriers, 29% (5 of 17) in stage I of intraepithelial carcinoma (CIN I), 37% (16 of 43) in stage II and III of intraepithelial carcinoma (CIN II-III), and 88% (15 of 17) in primary cervical carcinoma.

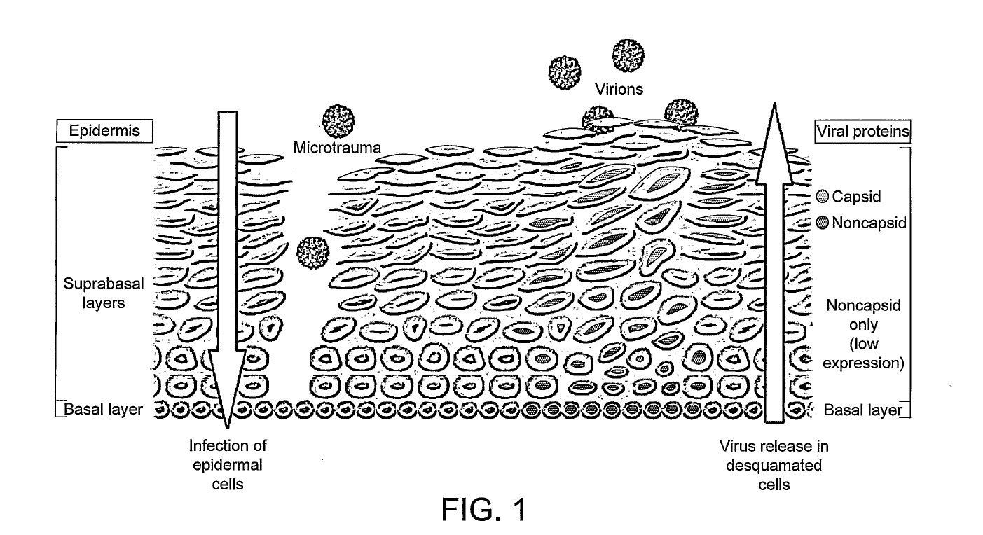

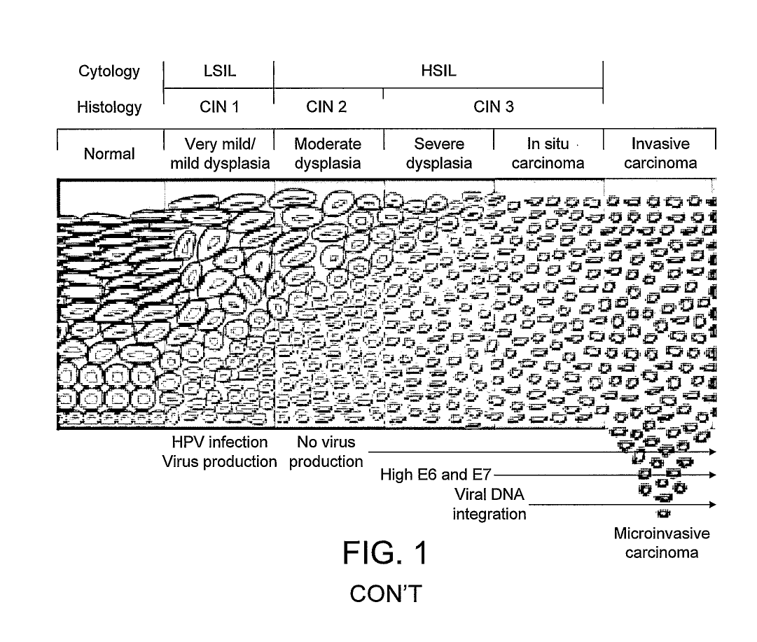

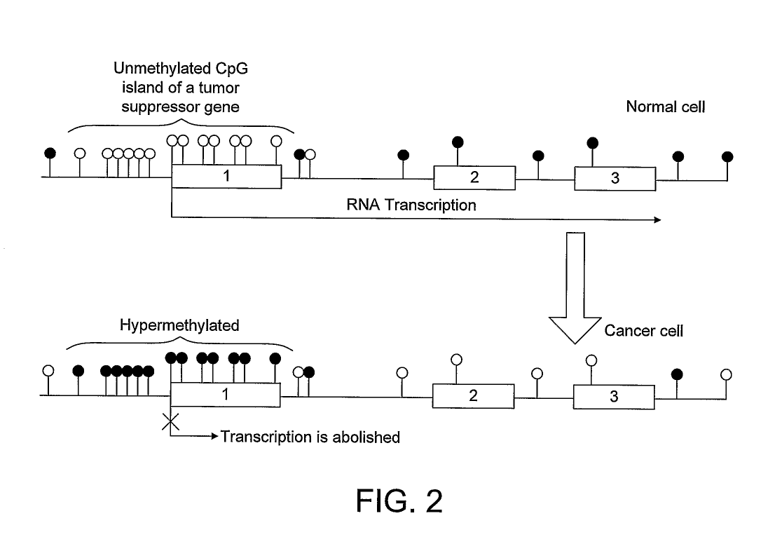

### DETAILED DESCRIPTION (EXPERIMENTAL PART)

The inventors have conducted analysis of the methylation pattern of all viral genomes in healthy virus-seropositive individuals and patients of different stage of the disease associated with the virus, and as well in human infected cell lines. The cells were purchased from the European Collection Cell Lines (ECACC) and The American Type Culture Collection (ATCC). The Tumour Bank Network operating in the CNIO has provided the human tissue under ideal conditions. The studies were carried out according to standardised procedures.

A subset of virus-infected representative cell lines and tumours were used: cell lines such as SiHa and CaSki, and tumours of the cervix in different progression stages.

The detection of the expression of viral oncogenic proteins was realized by Western blotting technique using commercially available antibodies, raised against E6 and E7.

To determine the methylation pattern of all EBV genes, Bisulfite Genomic Sequencing and Methylation-specific PCR (MSP) were used. Essentially, both detection methods use the C to T conversion by bisulfite when the cytosine is not methylated. The Primers used are described in Table 1 (Bisulfite genomic sequencing—SEQ ID NOs 1 to 42) and Table 2 (Methylation-specific PCR—SEQ ID NOs 43 to 46).

TABLE 1
Primers used for Bisulfite
Sequencing (BS). HPV16: Accession NC_001526

| Primers | Nucleotides Sequence (5′ → 3′) | |
|---|---|---|
| BS-hpv16-1 | Forward | 5′ TAATAATTTATGTATAAAATTAA |
| | | GGG 3′ |
| | Reverse | 5′ ATCCAAATATCTTTACTTTTCTT 3′ |
| BS-hpv16-2 | Forward | 5′ TGTTAAAAGTTATTGTGTTTTGAAG 3′ |
| | Reverse | 5′ ATCCAACTAAACCATCTATTTCAT 3′ |
| BS-hpv16-3 | Forward | 5′ ATGAAATAGATGGTTTAGTTGG 3′ |
| | Reverse | 5′ TCATCTAATATAACATCCCCTATT 3′ |
| BS-hpv16-4 | Forward | 5′ ATAGGGGATGTTATATTAGATG 3′ |
| | Reverse | 5′ AATATATCTTTCACTAACACCC 3′ |
| BS-hpv16-5 | Forward | 5′ TTAGTAATGTAAAGGTAGTAATGT |
| | | TAG 3′ |
| | Reverse | 5′ TTCCCCATAAACATACTAAAC 3′ |
| BS-hpv16-6 | Forward | 5′ GTGTTTTTAATGTGTATGATG 3′ |
| | Reverse | 5′ TACCTATATTAACTACACCATATA |
| | | AT 3′ |
| BS-hpv16-7a | Forward | 5′ TGTTGGTATAGATTTTAGGTGG 3′ |
| | Reverse | 5′ CAACCAATATTAACACCACTTAA 3′ |
| BS-hpv16-7b | Forward | 5′ ATAAGGTTAGAGAAATGGGATT 3′ |
| | Reverse | 5′ AACCCTCTACCACAATTACTAAT 3′ |
| BS-hpv16-8 | Forward | 5′ TAGTGGAAGTGTAGTTTGATGG 3′ |
| | Reverse | 5′ CACTATCCACTAAATCTCTATACA |
| | | ACA 3′ |
| BS-hpv16-9a | Forward | 5′ AAGTTGTTGTATAGAGATTTAGTG |
| | | GA 3′ |
| | Reverse | 5′ TAAACAAACACACAAAAACACA 3′ |
| BS-hpv16-9b | Forward | 5′ TTTTGTGTGTTTTTGTGTGTTT 3′ |
| | Reverse | 5′ AAACACCTAAACRCAAAAACTA 3′ |
| BS-hpv16-10a | Forward | 5′ TAGTAGTTTTTGYGTTTAGGTG 3′ |
| | Reverse | 5′ TTCAACAATAATTTTACCTTCAAC 3′ |
| BS-hpv16-10b | Forward | 5′ GGTTGAAGGTAAAATTATTGTTG 3′ |
| | Reverse | 5′ ACACCAACATCAATAAAACTAATTT 3′ |
| BS-hpv16-11 | Forward | 5' AGTAATTAGTAGTATATTTATATTA |
| | | GGG 3′ |
| | Reverse | 5' ATCATAATAATAATATACCTTAACA |
| | | CC 3′ |
| BS-hpv16-12 | Forward | 5′ GGATTATATGATATTTATGTAGAT |
| | | GAT 3' |
| | Reverse | 5′ ATCCAACTACAAATAATCTAAATA |
| | | TTC 3' |
| BS-hpv16-13 | Forward | 5′ TAAAGTATTAGGATTATAATATAG |
| | | GG 3′ |
| | Reverse | 5′ TCTATTATCCACACCTACATTTA 3′ |
| BS-hpv16-14a | Forward | 5′ GGTTTTGGTGTTATGGATTTTA 3′ |
| | Reverse | 5′ TTAATTACCCCAACAAATACCA 3′ |
| BS-hpv16-14b | Forward | 5′ AAAGGTTTTGGGTTTATTGTAA 3′ |
| | Reverse | 5′ ATTCCAATCCTCCAAAATAATA 3′ |
| BS-hpv16-15a | Forward | 5′ AAAGGAAAAGTTTTTTGTAGATTT 3′ |
| | Reverse | 5′ TAAATAACCACAACACAATTAATA |
| | | AA 3′ |
| BS-hpv16-15b | Forward | 5′ AGGATTGAAGGTTAAATTAAAATT 3′ |
| Reverse | Reverse | 5′ AACACATTTTATACCAAAAAACA 3′ |
| BS-hpv16-16 | Forward | 5′ TGTTTTTTGGTATAAAATGTGTT 3′ |
| | Reverse | 5′ CACACACCCATATACAATTTTA 3′ |

TABLE 2
Primer used for Methylation
Specific PCR (MSP) of the L2 gene
(M: methylation; U: no methylation)

| Primers | Nucleotides Sequence. (5′ → 3′) | |
|---|---|---|
| MSP-hpv16-4-M | Forward5′ TGCGATATAAACGTTTTGTAAAAC 3′ | |
| | Reverse | 5′ AATATACCCAATACGTCCGC 3′ |
| MSP-hpv16-4-U | Forward | 5′ TAATGTGATATAAATGTTTTGTAA |
| | | AAT 3′ |
| | Reverse | 5′ AAATATACCCAATACATCCACCT 3′ |

Results and Discussion

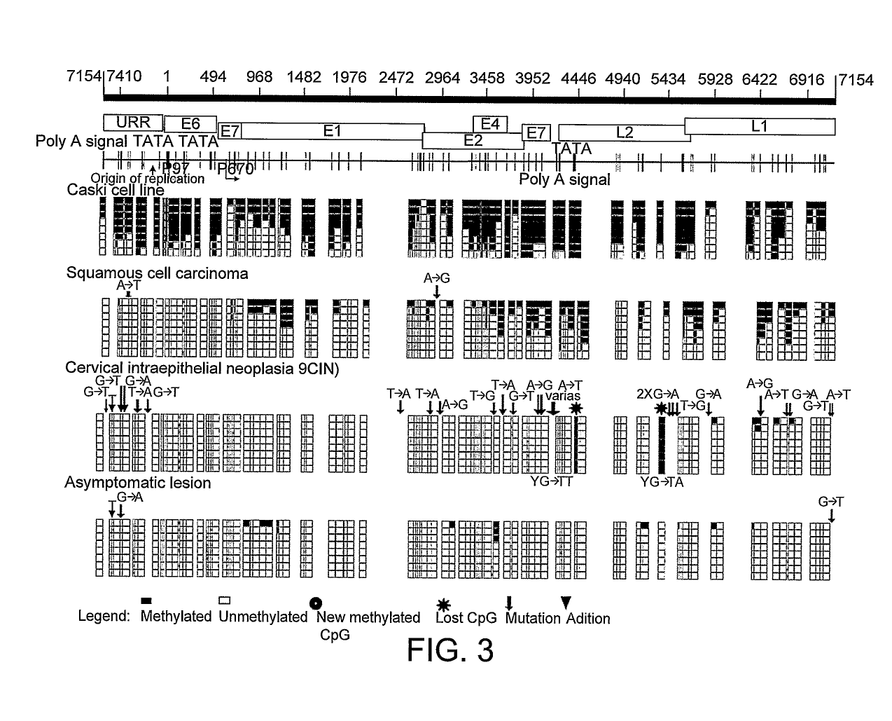

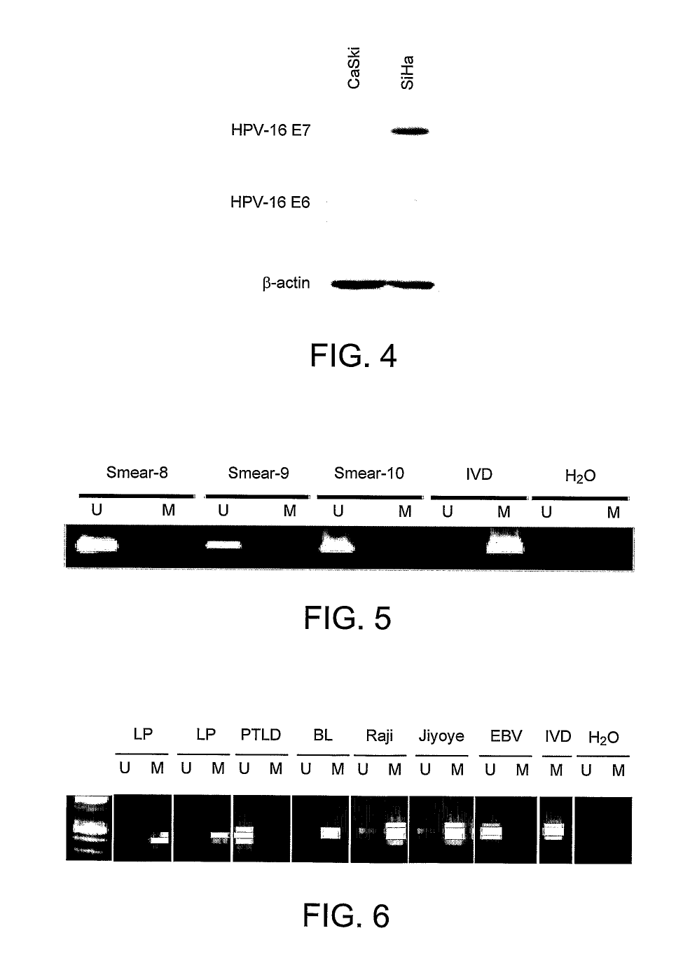

Our results in HPV-16 analyzing the whole genome of the virus provide us with the first complete DNA methylome of a virus. It shows DNA hypomethylation in asymptomatic lesions and CIN compared to primary squamous cell carcinomas and the cervical cancer cell lines, such as CaSki (FIG. 3). Our work using bisulfite genomic sequencing gives as well very useful information about genetic alterations, making the analysis very complete from a genetic and an epigenetic point of view (FIG. 3). In our results we can detect as well the protein expression of E6 and E7 protein products in Caski and SiHa cell lines, (FIG. 4).

Finally, following identification of the complete DNA methylomes of HPV16 in asymptomatic patients, CIN and cervical tumors by bisulfite genomic sequencing, we have gone one further step by developing a Methylation-Specific PCR (MSP) assay to detect the presence of CpG methylation at critical differentially methylated regions of the HPV16 genome. The use of MSP-HPV16 permits the detection of DNA methylation of the virus in any large collection of samples (n>100). Representative results are shown in FIG. 5.

Although great efforts are currently being made to prevent cervical cancer, including developing prophylactic vaccines and improving early detection; the widespread nature of HPV infection requires greater understanding of both the HPV life cycle and the mechanisms underlying HPV-induced carcinogenesis.

The study of aberrant hypermethylation of various genes in combination with the methylation pattern of human papilloma viruses acts as a useful tool for the screening and prognosis of cervical cancer.

Over 100 different papilloma family members exist and, in humans, are responsible for a variety of benign proliferations, however, infection with two specific high-risk HPVs, HPV-16 and HPV-18, are associated with approximately 90% of uterine cervical cancers, more than 50% of other anogenital tumors and a small percentage of head and neck tumors (zur Hausen 1996 BBA). HPV particles consist of 8000 base-pair (bp) long circular DNA molecules wrapped into a protein shell that is composed of two molecules (L1 and L2). The genome has the coding capacity for these two proteins and at least six so-called early proteins (E1, E2, E4-E7) that are necessary for the replication of the viral DNA and for the assembly of newly produced virus particles within the infected cells. Both sets of genes are separated by an upstream regulatory region (URR) of about 1000 by that does not code for proteins but contains cis-elements required for regulation of gene expression, replication of the genome and its packaging into virus particles. Two of the viral proteins, E6 and E7, are essential for HPV-mediated cellular transformation (Ref). E6 and E7 interfere with many cellular processes overriding signaling pathways and cell cycle control by altering the function of basal transcription factors, p53 and Rb, among other target proteins.

The natural history of the disease is intriguing: only a minority of cervical lesions infected with HPV inevitable progress to cancer. Given the prevalence of genital HPV types in the general population (and 20-40% amongst young women depending on geographical location) and the high life-time risk of infection (estimated at 80%), incidence of cervical cancer is low (approx. 0.03% in the absence of screening). Thus, the development of cervical cancer occurs in a few women who cannot resolve their infection and who maintain persistent active infection for years or decades following initial exposure. However, the molecular reasons for controlling the infection or instead progressing in the tumorigenesis pathway are largely unknown. The complete HPV DNA methylomes obtained in our study might provide important clues to understand the described process.

We have first sequenced the whole DNA methylome of the HPV16 virus (110 CpGs in 7904 nucleotides) in a collection of human cervical samples corresponding to the different progressive stages of the disease: asymptomatic carriers (n=5), premalignant disease (the so called Intraepithelial carcinoma, CIN) (n=8) and the primary cervical carcinomas (n=6). We have also completed the HPV16 DNA methylome of two long established cervical cancer cell lines (CaSki and SiHa). We have discovered that the HPV16 DNA methylome undergo a progressive increase in its DNA methylation content from the women carriers of the virus but without any clinical symtomatology, to the early pre-tumorigenic lesions, to end with the full-blown primary cervical carcinomas. In this sequence of events, the HPV16 DNA methylomes of the aggressive cervical cancer cell lines CaSki and SiHA are the ones demonstrating the higher DNA methylation levels. We have developed an unsupervised clustering analyses for the nineteen obtained HPV16 DNA methylomes that also distinguished the four different biological groups: carriers, premalignancies, primary tumors and cancer cell lines.

The observed dynamic changes undergoing the viral DNA methylome in the tumorigenic process were underlined when we studied cultured primary human foreskin keratinocytes transfected with the entire genome of HPV16 (Steenbergen Oncogene 1996). The HPV16 DNA methylome from the pre-immortal keratynocytes was almost completely unmethylated, whilst the immortal cells presented a densely methylated viral genome. Most important, genetic or nucleotidic changes in the HPV16 genome were not observed. This observation was a permanent feature in our studies: because our DNA methylation analyses by bisulfite genomic sequencing also allows the determination of putative nucleotide changes (such as point mutations, insertions or deletions), we were able to assess the contribution of these genetic changes in the virus itself to the progression of the disease. We did not observe any particular viral nucleotide change associated with the natural history of cervical tumorigenesis.

The HPV16 genome does not encode for any gene of the DNA methylation machinery, such as DNA methyltransferases (DNMTs). Thus, the viral genome is methylated by the host human cellular DNMTs. Because the integration of HPV16 in the human genome occurs in most cervical tumors and a significant proportion of premalignant lesions, we can consider that human DNMTs might recognize these sequences as “foreign” DNA that needs to be methylated. Indeed in our study, we have confirmed by FISH analyses that the HPV16 genome is integrated in the host cell DNA in CaSki and SiHa. Most important, confocal studies demonstrate the co-localization of the integrated viral genome and the 5-methylcytosine DNA staining. Depletion of DNMT1, DNMT3b and both DNMTs by short interference RNA in CaSki cells indeed cause a DNA hypomethylation of the HPV16 genome. Interestingly, the HPV16 genome is not a mere passive espectator of the process but might actively participate by recruiting DNMTs (camouflage) using the viral oncoprotein E7 (Burgers W A Oncogene 2007).

The described HPV16 DNA methylomes also reflect the proposed expression patterns of the virus in cervical tumorigenesis, characterized by a significant overexpression of E6 and E7 in the carcinomas. Our sequencing effort shows that for a subset of cases across the spectrum of stages there are genomic viral regions that are deleted or cannot be amplified by PCR (grey areas in the cluster). This event is particularly observed for the E1/E2 region. The protein E2 is an inhibitor of cell proliferation by regulating the viral URR, repressing the E6 and E7 oncoproteins and ultimately causing cell cycle arrest at G2 (Frattini 1997 EMBO J). Thus, the genomic loss of E2 could contribute to the progression of the disease. However, the described HPV16 DNA methylomes show that there are many cases where there are not ruptures at the E2 locus. We observe instead methylation of the E2-binding sites at the URR region that are associated with high level of expression of the oncoproteins E6 and E7. Most important, we show by chromatin immunoprecipitation (ChIP) that the E2 viral protein cannot bind to the methylated E2-binding sites at the URR region, and thus leads to E6 and E7 overexpression. Finally, the induction of hypomethylation events in the E2-binding sites at the URR region by a DNA demethylating agent (5-aza-2′-deoxycytidine) or short interference RNA of the DNMTs induces a recruitment of E2 to its URR binding sites and a marked reduction of E6 and E7 expression.

Overall, the outlined DNA methylomes of HPV16 supports the existence of an adaptative DNA methylation pattern of the viral genome with an increased methylated-CpG content in the progression of the disease originated from a crosstalk between the viral and the host genomes. We have gone one step further to confirm these results in a large collection (n=87) of human primary samples from the different stages of cervical carcinogenesis using the technique of methylation-specific PCR for the L2 region of the virus. We have indeed observed the progressive presence of hypermethylation at the L2 locus in tumorigenesis: none (0 of 10) in asymptomatic carriers, 29% (5 of 17) in stage I of intraepithelial carcinoma (CIN I), 37% (16 of 43) in stage II and III of intraepithelial carcinoma (CIN II-III), and 88% (15 of 17) in primary cervical carcinoma. Results are presented in FIG. 6.

### REFERENCES

1. Badal S, et al. Virology, 324: 483-492 (2004).

2. Bosch F X, et al. Journal of Clinical Pathology, 54: 163-175 (2001).

3. Butel J S. Carcinogenesis, 21: 405-426 (2000).

4. Davies P, et al. Int. J. Cancer, 118: 791-796 (2006).

5. De Villiers E M, et al. Virology, 324: 17-27 (2004).

6. Dong S M, et al. Clinical Cancer Research, 7: 1982-1986 (2001).

7. Esteller M. Annu. Rev. Pharmacol. Toxicol., 45: 629-656 (2005).

8. Gatza M L, et al. Environmental and Molecular Mutagenesis, 45: 304-325 (2005).

9. Gillinson M L. Seminars in Oncology, 31: 744-754 (2004).

10. Hebner C M, et al. Reviews in Medical Virology, 16: 83-97 (2006).

11. Kalantary M, et al. Journal of Virology, 78: 12762-12772 (2004).

12. Li H P, et al. Cell Research, 15: 262-271 (2005).

13. Lowy D R, et al. The Journal of Clinical Investigation, 116: 1167-1173 (2006).

14. Pfister, H. Journal of the National Cancer Institute Monographs, 31: 52-56 (2003).

15. Turan T, et al. Virology, 349: 175-183 (2006).

16. Virmani A K, et al. Clinical Cancer Research, 7: 584-589 (2001).

17. Zur Hausen H, et al. Nature Reviews, Cancer, 2: 342-350 (2002).

1. A method of diagnosing or monitoring the progression of or otherwise staging a disease caused by a human papillomavirus (HPV) infection in a test sample obtained from a subject comprising determining the methylation status of a HPV genome wherein the presence of hypermethylation of the HPV genome indicates a positive diagnosis of the disease or an increased level of methylation of the HPV genome indicates the progression of the disease to a more advanced form.

2. A method of monitoring the progression of a disease caused by a human papillomavirus (HPV) infection in response to a treatment directed against the disease in a test sample obtained from a subject comprising determining the methylation status of a HPV genome before and following treatment of the subject wherein the presence of decreased methylation of the HPV genome following treatment indicates a positive effect of the treatment on the disease in terms of successfully inhibiting disease progression.

3. The method of claim 2 wherein the presence of equal methylation before and following treatment indicates that the treatment has been successful in preventing progression of the disease.

4. (canceled)

5. The method of claim 1 wherein the determined methylation status is compared to a control.

6. (canceled)

7. (canceled)

8. The method of claim 1 wherein the disease comprises cancer.

9. The method of claim 8 wherein the cancer is cervical cancer.

10. The method of claim 1 which is utilised in order to stage the disease as one of HPV carrier, pre-malignancy or primary tumour depending upon the methylation status.

11. The method of claim 10 wherein hypomethylation of the HPV genome indicates a HPV carrier and hypermethylation indicates a primary tumour.

12. The method of claim 10 wherein an intermediate methylation status between hypomethylation and hypermethylation indicates pre-malignancy.

13.-16. (canceled)

17. The method of claim 1 wherein the methylation status of the entire HPV genome is determined.

18. The method of claim 1 which comprises determination of the methylation status of a plurality of CpG residues in the nucleotide sequences which can be sequenced using the primers comprising the nucleotide sequences set forth as SEQ ID NOs 1 to 42 or amplified using the primers comprising the nucleotide sequences as set forth as SEQ ID NOs 43 to 46.

19. The method of claim 1 wherein determining the methylation status of the HPV genome comprises determining the methylation status of the L2 gene.

20. The method of claim 1 wherein determining the methylation status of the HPV genome comprises determining the methylation status of the E2 binding sites in the upstream regulatory region.

21. The method of claim 1 wherein determining the methylation status of the HPV genome comprises determining whether E6 or E7 is overexpressed.

22. The method of claim 1 wherein the HPV is HPV16.

23. A primer for use in bisulphite sequencing of a HPV genome selected from the primers comprising the nucleotide sequences set forth as SEQ ID NO: 1 to 42 and functional derivatives thereof which retain functionality in bisulphite sequencing.

24. A primer for use in methylation specific PCR (MSP) to determine the methylation status of the L2 gene of a HPV genome selected from the primers comprising the nucleotide sequences set forth as SEQ ID NO: 43 to 46 and functional derivatives thereof which retain functionality in MSP.

25. (canceled)

26. A kit comprising a primer pair selected from the primers claimed in claim 23.

27.-29. (canceled)

30. A method of diagnosing or monitoring the progression of or otherwise staging a disease caused by a human papillomavirus (HPV) infection in a test sample obtained from a subject comprising determining the expression levels of E6 or E7 wherein the presence of overexpression of E6 or E7 indicates a positive diagnosis of the disease or an increased level of expression of E6 or E7 indicates the progression of the disease to a more advanced form.

31. A method of diagnosing squamous cell carcinoma in a test sample obtained from a subject comprising determining the methylation status of a HPV-16 genome wherein the presence of hypermethylation of the HPV-16 genome indicates a positive diagnosis of squamous cell carcinoma.

32. A method of distinguishing squamous cell carcinoma and cervical intraepithelial neoplasia in a test sample obtained from a subject comprising determining the methylation status of a HPV-16 genome wherein the presence of hypermethylation of the HPV-16 genome indicates the presence of squamous cell carcinoma, whereas hypomethylation of the HPV-16 genome indicates the present of cervical intraepithelial neoplasia.

33. (canceled)

34. The method of claim 2 wherein the determined methylation status is compared to a control.

35. The method of claim 2 wherein the disease comprises cancer.

36. The method of claim 2 which is utilised in order to stage the disease as one of HPV carrier, pre-malignancy or primary tumour depending upon the methylation status.

37. The method of claim 2 wherein the methylation status of the entire HPV genome is determined.

38. The method of claim 2 which comprises determination of the methylation status of a plurality of CpG residues in the nucleotide sequences which can be sequenced using the primers comprising the nucleotide sequences set forth as SEQ ID NOs 1 to 42 or amplified using the primers comprising the nucleotide sequences as set forth as SEQ ID NOs 43 to 46.

39. The method of claim 2 wherein determining the methylation status of the HPV genome comprises determining the methylation status of the L2 gene.

40. The method of claim 2 wherein determining the methylation status of the HPV genome comprises determining the methylation status of the E2 binding sites in the upstream regulatory region.

41. The method of claim 2 wherein determining the methylation status of the HPV genome comprises determining whether E6 or E7 is overexpressed.

42. The method of claim 2 wherein the HPV is HPV16.

43. A kit comprising a primer pair selected from the primers claimed in claim 24.
